(19) United States
(12) Patent Application Publication
(10) Pub. No.: US 2022/0079855 A1
(43) Pub. Date: Mar. 17, 2022

(54) GINGSENG-BASED COSMETIC COMPOSITION

(71) Applicant: L'OREAL, Paris (FR)

(72) Inventors: Laurence VESPERINI-BOUADJENEK, Chevilly Larue (FR); Sonia EYRAUD, Chevilly Larue (FR); Isabelle BOSSANT, Chevilly Larue (FR)

(73) Assignee: L'OREAL, Paris (FR)

(21) Appl. No.: 17/417,612

(22) PCT Filed: Dec. 24, 2019
(86) PCT No.: PCT/EP2019/087027

§ 371 Date: Jun. 23, 2021

(30) Foreign Application Priority Data
Dec. 24, 2018 (FR) 1874124

(51) Int. Cl. A61K 8/368; A61K 8/9789; A61K 8/06; A61Q 19/00

(52) U.S. Cl. CPC A61K 8/368; A61K 8/9789; A61K 8/062; A61K 2800/805; A61Q 19/00; A61K 2800/10; A61K 2800/591; A61K 8/064

(57) ABSTRACT

The present invention relates mainly to a composition, in particular a cosmetic composition, intended for skincare, comprising water and at least: —from 0.001% to 10% by weight of 5-n-octanoylsalicylic acid or a salt thereof relative to the total weight of said composition —from 0.001% to 10% by weight, relative to the total weight thereof, of a plant active agent containing at least 0.05% by weight of ginsenoside Rg3 relative to the total weight thereof.

### TECHNICAL FIELD

The present invention relates to the field of compositions intended for skincare.

More particularly it targets a composition, in particular a cosmetic composition, for topical administration comprising a combination of active agents intended to stimulate the radiance of the skin, to improve the smooth appearance thereof, by favouring notably the desquamation of the skin.

### PRIOR ART

It is recalled that desquamation is a natural phenomenon associated with the fact that the epidermis, which constitutes the upper layer of the skin, is constantly being regenerated. This epidermis is constituted of several layers of cells, the deepest of which is the basal layer constituted of undifferentiated cells. Over time, these cells will differentiate and migrate towards the surface of the epidermis, making up the various layers thereof, until they form, at the surface of the epidermis, the corneocytes. The stacking of these corneocytes constitutes the horny layer which is responsible for the barrier function of the epidermis. In the course of the normal desquamation process, the most superficial corneocytes detach from the surface of the epidermis. This loss at the surface is compensated for by the migration of cells from the basal layer towards the surface of the epidermis. There is perpetual renewal of the skin. Desquamation thus makes it possible to stimulate epidermal renewal and to restore or increase a physiologically healthy state of the skin.

Furthermore, it is known that the desquamation process can be influenced by exogenous factors (examples: UV radiation, pollution, and the like) and/or endogenous factors (examples: hormonal changes, age, and the like) and can result in particular in a slowing of this epidermal renewal and consequently give rise to an ageing of the skin and/or a thickening of the horny layer, such as the formation of callosities.

Consequently, the stimulation of the skin desquamation mechanism is an effective means for improving the radiance of the complexion, reducing surface irregularities and smoothing the skin, or for promoting the cleansing action and the removal of the dead cells at the surface of the body and scalp.

Generally, desquamating agents act by facilitating the removal of the dead cells located at the surface of the horny layer of the epidermis. Mention may in particular be made, among these cosmetic agents promoting desquamation, that is to say the removal of the “dead” cells located at the surface of the horny layer of the epidermis, of α-hydroxy acids (AHAs), such as lactic acid or glycolic acid, or β-hydroxy acids (BHAs), such as salicylic acid. These active agents bring about, by topical application, a desquamation visible after a few days. In particular, capryloyl-salicylic acid is recognized as being a particularly effective desquamating agent (FR0752995).

### SUMMARY OF THE INVENTION

One objective of the present invention is specifically to propose a means for stimulating the efficacy of a desquamating active agent such as capryloyl-salicylic acid.

Another objective of the invention is to propose a particularly effective composition for stimulating epidermal renewal, in particular through the regulation of the desquamation of the skin.

Another objective of the invention is to propose a composition for promoting, restoring or increasing the homeostasis of the epidermis, in particular through the regulation of the desquamation of the skin.

Another objective of the present invention is to propose a composition for treating and/or preventing an aesthetic skin defect, resulting from, or being associated with, a deterioration of the desquamation of the skin. Such defects may in particular be a deterioration of the complexion or of the surface of the skin.

Another objective of the present invention is to propose a composition for promoting and/or increasing the hydration of the skin, and in particular for preventing and/or treating dry skin or signs of skin dryness, by acting, in particular, through the regulation of the desquamation of the skin.

Another objective of the present invention is to propose a composition for treating and/or preventing a pathological skin disorder resulting from, or being associated with, a deterioration of the desquamation of the skin, such as atopic dermatitis, ichthyosis, hyperkeratosis, or psoriasis.

More specifically, the present invention relates to a composition, in particular a cosmetic composition, and more particularly intended for caring for the skin, in particular of the face, comprising at least:
from 0.001% to 10% by weight of 5-n-octanoylsalicylic acid or a salt thereof relative to the total weight of said composition
from 0.001% to 10% by weight, relative to the total weight thereof, of a plant active agent containing at least 0.05% by weight of ginsenoside Rg3 relative to the total weight of said active agent, and
water.

Preferably, said plant active agent is red ginseng.

Ginseng, also referred to as Ginseng Radix which is the root or rhizome of ginseng, has been used in Eastern Asia for 2000 years as a nutritional tonic agent, promoting health and longevity. Pharmacological effects, in particular with regard to arteriosclerosis, and for treatment of hyperlipidaemia, treatment of hepatic insufficiency, improvement of the hepatic function, protection against radiation, boosting immunity, for improvement of the cerebral function, and also antithrombotic, antistress, antidiabetic, antihypertensive and antitumour effects, are also reported nowadays for ginseng.

As a general rule, ginseng is used in the form of fresh ginseng not yet dried, of white ginseng prepared by drying the fresh ginseng at a standard temperature or of red ginseng prepared by steam treatment and by drying fresh ginseng at a temperature of 98° C. to 120° C. for 1 to 3 hours.

Against all expectations, the inventors observed that among these white and red ginsengs, only the combination of red ginseng proves particularly beneficial with respect to the efficacy of 5-n-octanoylsalicylic acid or salts thereof. This unexpected effect is in particular illustrated in the examples which follow.

However, it is known that red ginseng does not have the same composition as white ginseng. This difference is in particular linked to the generation of specific compounds during the heat treatment of white ginseng to form red ginseng. Thus the ginsenosides Rh2, Rs1, Rs2, Rg3, Rg2, Rh1, etc. and the polyacetylene compound such as panaxytriol are only present in red ginseng. In particular, the amount of Rg3, present only in trace amounts or even completely absent in white ginseng, increases significantly in red ginseng depending on the duration and temperature of the heat treatment considered in order to form it.

Admittedly, cosmetic compositions intended for skincare or haircare, and that use an extract of ginseng and notably of red ginseng and in particular of red ginseng have already been described for example in Korean patent no. 2002-0040707, Korean patent no. 1996-0020985, the publication of Japanese patent no. 62-93217, U.S. Pat. No. 5,182,373. However, to the knowledge of the inventors, the beneficial effect of red ginseng with regard to the efficacy of 5-n-octanoylsalicylic acid has never been identified.

The present invention also relates to a cosmetic process for stimulating epidermal renewal, in particular through the regulation of desquamation, comprising at least the topical application of a composition.

The present invention also relates to a cosmetic process for promoting, restoring or increasing the homeostasis of the epidermis, in particular through the regulation of the desquamation, comprising at least the topical application of a composition according to the invention.

The present invention also relates to a cosmetic process for treating and/or preventing an unsightly deterioration of the complexion or of the surface of the skin, by acting, in particular, on the regulation of the desquamation of the skin, comprising at least the topical application of a composition according to the invention.

Another objective of the present invention is to propose a cosmetic process for promoting and/or increasing the hydration of the skin, and in particular for preventing and/or treating dry skin or signs of skin dryness, by acting, in particular, on the regulation of the desquamation of the skin comprising at least the topical application of a composition according to the invention.

### DETAILED DESCRIPTION

5-n-Octanoylsalicylic acid, also referred to as caproyl salicylic acid as INCI name or 2-hydroxy-5-(1-oxooctyl)benzoic acid, has the following structure:

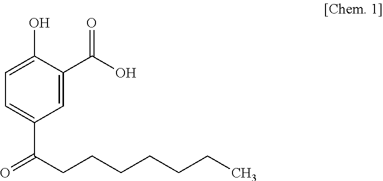 [SMILES: CCCCCCCC(=O)c1ccc(O)c(C(=O)O)c1]

Generally, the composition in accordance with the invention may comprise from 0.001% to 10%, in particular from 0.05% to 2.0%, better still from 0.1% to 1.0% of 5-n-octanoylsalicylic acid or a salt thereof relative to the total weight of the composition.

The salts of 5-n-octanoylsalicylic acid may be obtained by salification with a mineral or organic base. Examples of mineral bases that may be mentioned include alkali metal or alkaline-earth metal hydroxides, for instance sodium hydroxide or potassium hydroxide, or ammonium hydroxide.

As stated previously, this 5-n-octanoylsalicylic acid or a salt thereof is combined with a plant active agent containing at least 0.05% by weight of ginsenoside Rg3 relative to the total weight thereof.

This specific ginsenoside, Rg3, is known for only being present in a significant amount in red ginseng which is itself obtained by heat treatment of white ginseng.

The document J. Ginseng Res. Vol 29, No. 1, 1-18 (2005) very particularly documents this difference in composition between the white and red ginsengs. In contrast to red ginseng, white ginseng contains this ginsenoside Rg3 only in trace amounts.

Ginseng is a perennial plant with deciduous leaves belonging to the family of Araliaceae.

The genus of ginseng very particularly suitable for the invention is Panax ginseng. As representatives of this genus, mention may in particular be made of the species Panax ginseng C. A. Meyer cultivated in Korea, Japan, China, Russia and Germany; Panax quinquefolius L. (American ginseng), Panax japonicus C. A. Meyer (Japanese ginseng), cultivated in Japan; Panax notoginseng (Burkill.) F. H. Chen (Sanchi ginseng) that grows in China; Panax trifolius L. (dwarf ginseng), Panax major Ting; Panax omeiensis J. Wen; Panax pseudoginseng Wallich, Panax sinensis J. Wen; Panax stipuleanatus H. T. Tsai & K. M. Feng; Panax wangianus Sun; Panax zingiberensis C. Y. Wu & K. M. Feng (11) and Panax vietnamensis Ha and Grushv.

Panax ginseng is generally harvested after growing for 4 to 6 years and it is classified into three types depending on its treatment: a) fresh ginseng (aged less than 4 years; consumable in the fresh state); b) white ginseng (aged 4 to 6 years; dried after peeling); and c) red ginseng (harvested at the age of 6 years, then steamed and dried).

As specified above, the form of these ginsengs that is suitable for the invention is the one containing the ginsenoside Rg3, and therefore red ginseng.

There are commercial formulae of red ginseng, such as for example the Red Ginseng Extract distributed by CRODAROM, or else the Red Ginseng Active R distributed by Natural Solution.

The plant active agent used according to the invention is generally in liquid or powder form.

Advantageously, the plant active agent considered according to the invention contains at least 0.1%, preferably at least 0.2% and more preferentially at least 0.5% or even more than 1% by weight of ginsenoside Rg3 relative to the total weight thereof.

This content may be determined by HPLC-UV analysis and in particular according to the methodology described in the article Samukawa Yagaku Zasshi. 115, 241-249.

This ginsenoside Rg3 is in particular present in the plant active agent in combination with at least one ginsenoside chosen from the ginsenosides Rh2, Rs1, Rs2, Rg2 and Rh1.

According to another preferred embodiment, a composition according to the invention contains at least 0.05% by weight of plant active agent, in particular at least 0.1%, preferably at least 0.3% or even at least 0.5% by weight of plant active agent, and in particular of red ginseng.

Within the meaning of the invention, the plant active agent denotes the active material as is which may be used in an isolated form or formulated in solution. It may be for example, like in the examples which follow, 2% of an aqueous solution containing 5% of active material.

According to another preferred embodiment, a composition according to the invention contains this plant active agent and 5-n-octanoylsalicylic acid or a salt thereof in a weight ratio of plant active agent at least equal to 0.3, preferably greater than 1 and more preferentially greater than 1.5.

In particular, a composition according to the invention may contain 0.3% by weight of 5-n-octanoylsalicylic acid or a salt thereof per 0.1% by weight of active material of said plant active agent, or even 0.3% by weight of 5-n-octanoylsalicylic acid or a salt thereof per 0.5% by weight of active material of said plant active agent.

As stated previously, a composition according to the invention further contains water.

In other words, a composition according to the invention is not anhydrous.

The amount of water is not limited, and may range from 50% to 99% by weight, preferably from 55% to 95% by weight and more preferably from 70% to 90% by weight relative to the total weight of the composition.

According to a particular embodiment of the invention, the composition according to the present invention may further comprise at least one C₂-C₄ monoalcohol.

The C₂-C₄ monoalcohols comprise a hydroxy group and from 2 to 4 carbon atoms. Use may be made of ethanol, propanol, isopropanol, 1-butanol or isobutanol, and mixtures thereof. The C₂-C₄ monoalcohol is preferably ethanol.

The concentration of C₂-C₄ monoalcohol may range from 0.1% to 30% by weight, preferably from 0.5% to 15% by weight, preferably from 1% to 10% by weight relative to the total weight of the composition.

More generally, the compositions according to the invention may be characterized as containing a physiologically acceptable medium, i.e. a medium that is suitable for the topical administration of a composition, i.e. that is compatible with the skin.

According to the invention, a physiologically acceptable medium is preferentially a cosmetically acceptable medium, i.e. a medium which is free of any odour or unpleasant appearance and which is entirely compatible with the topical administration route.

The term “skin” is intended to mean all of the skin of the body, and preferably the skin of the face, neckline, neck, arms and forearms, or even more preferably still the skin of the face, especially of the forehead, nose, cheeks, chin and area around the eyes.

Thus, a composition of the invention may be a cosmetic composition (i.e. intended for cosmetic use) or a dermatological composition. Preferentially, according to the invention, the composition is a cosmetic composition and even more preferentially a cosmetic composition for topical application.

The compositions according to the invention may be in the form of aqueous solutions, aqueous-alcoholic solutions, oil-in-water (O/W) emulsions, water-in-oil (W/O) emulsions or multiple emulsions (triple: W/O/W or O/W/O), or aqueous gels. These compositions are prepared according to the usual methods.

The composition may comprise at least one oil.

As oils that may be used, examples that may be mentioned include:
hydrocarbon-based oils of plant origin, such as liquid fatty acid triglycerides including from 4 to 10 carbon atoms, for instance heptanoic or octanoic acid triglycerides, or alternatively, for example, sunflower oil, corn oil, soybean oil, marrow oil, grapeseed oil, sesame seed oil, hazelnut oil, apricot oil, macadamia oil, arara oil, castor oil, avocado oil, caprylic/capric acid triglycerides, for instance those sold by the company Stearineries Dubois or those sold under the names Miglyol 810, 812 and 818 by the company Dynamit Nobel, jojoba oil and shea butter oil;
synthetic esters and ethers, especially of fatty acids, for instance the oils of formulae R₁COOR₂ and R₁OR₂ in which R₁ represents a fatty acid residue containing from 8 to 29 carbon atoms and R₂ represents a branched or unbranched hydrocarbon-based chain containing from 3 to 30 carbon atoms, for instance purcellin oil, isononyl isononanoate, isopropyl myristate, 2-ethylhexyl palmitate, 2-octyldodecyl stearate, 2-octyldodecyl erucate or isostearyl isostearate; hydroxylated esters, for instance isostearyl lactate, octyl hydroxystearate, octyldodecyl hydroxystearate, diisostearyl malate, triisocetyl citrate, and fatty alcohol heptanoates, octanoates and decanoates; polyol esters, for instance propylene glycol dioctanoate, neopentyl glycol diheptanoate and diethylene glycol diisononanoate; and pentaerythritol esters, for instance pentaerythrityl tetraisostearate;
linear or branched hydrocarbons of mineral or synthetic origin, such as volatile or non-volatile liquid paraffins, and derivatives thereof, petroleum jelly, polydecenes, and hydrogenated polyisobutene such as Parleam oil;
fatty alcohols containing from 8 to 26 carbon atoms, such as cetyl alcohol, stearyl alcohol and a mixture thereof (cetylstearyl alcohol), octyldodecanol, 2-butyloctanol, 2-hexyldecanol, 2-undecylpentadecanol, oleyl alcohol or linoleyl alcohol;
silicone oils, for instance volatile or non-volatile polymethylsiloxanes (PDMS) with a linear or cyclic silicone chain, which are liquid or pasty at room temperature, notably cyclopolydimethylsiloxanes (cyclomethicones) such as cyclohexasiloxane; polydimethylsiloxanes including alkyl, alkoxy or phenyl groups, which are pendent or at the end of a silicone chain, these groups containing from 2 to 24 carbon atoms; phenyl silicones, for instance phenyl trimethicones, phenyl dimethicones, phenyltrimethylsiloxydiphenylsiloxanes, diphenyl dimethicones, diphenylmethyldiphenyltrisiloxanes or 2-phenylethyl trimethylsiloxy silicates, and polymethylphenylsiloxanes; and
mixtures thereof.

In the list of oils mentioned above, the term “hydrocarbon-based oil” means any oil predominantly including carbon and hydrogen atoms, and possibly ester, ether, fluoro, carboxylic acid and/or alcohol groups.

The composition according to the invention may further comprise substances that are solid at room temperature (25° C.), for instance fatty acids comprising from 8 to 30 carbon atoms, such as stearic acid, lauric acid, palmitic acid and oleic acid; waxes such as lanolin, beeswax, carnauba wax or candelilla wax, paraffin waxes, microcrystalline waxes, ceresin or ozokerite, and synthetic waxes such as polyethylene waxes and Fischer-Tropsch waxes.

These fatty substances may be chosen in a varied manner by a person skilled in the art in order to prepare a composition having the desired properties, for example in terms of consistency or texture.

According to one particular embodiment of the invention, the composition according to the invention is a water-in-oil (W/O) or oil-in-water (O/W) emulsion, and preferably an oil-in-water emulsion.

The proportion of the oily phase of a composition according to the invention may range from 1% to 80% by weight and preferably from 5% to 50% by weight relative to the total weight of the composition.

Such emulsions generally contain at least one emulsifier chosen especially from amphoteric, anionic, cationic and nonionic emulsifiers, used alone or as a mixture, and optionally a co-emulsifier. The emulsifiers are chosen in an appropriate manner according to the emulsion to be obtained (W/O or O/W emulsion).

The emulsifier and the co-emulsifier are generally present in the composition in a proportion ranging from 0.3% to 30% by weight and preferably from 0.5% to 20% by weight relative to the total weight of the composition.

For the W/O emulsions, examples of emulsifiers that may be mentioned include dimethicone copolyols, such as the mixture of cyclomethicone and dimethicone copolyol sold under the name DC 5225 C by the company Dow Corning, and alkyl dimethicone copolyols such as the lauryl methicone copolyol sold under the name Dow Corning 5200 Formulation Aid by the company Dow Corning, and the cetyl dimethicone copolyol sold under the name Abil EM 90® by the company Goldschmidt.

For the O/W emulsions, examples of emulsifiers that may be mentioned include nonionic emulsifiers such as oxyalkylenated (more particularly polyoxyethylenated) fatty acid esters of glycerol; oxyalkylenated fatty acid esters of sorbitan; oxyalkylenated (oxyethylenated and/or oxypropylenated) fatty acid esters; oxyalkylenated (oxyethylenated and/or oxypropylenated) fatty alcohol ethers; sugar esters such as sucrose stearate; and mixtures thereof, such as the mixture of glyceryl stearate and PEG-100 stearate.

The composition may be an aqueous gel, and may especially comprise common aqueous gelling agents.

In a known manner, the composition according to the invention may also contain adjuvants that are common in the cosmetic or dermatological field, such as thickeners, film-forming polymers, preservatives, fragrances, fillers, UV-screening agents, bactericides, odour absorbers, colorants, plant extracts, vitamins, polyols such as glycerol or diglycerol, sugars such as sorbitol, additional cosmetic and dermatological active agents, and salts. The amounts of these various adjuvants are those conventionally used in the field under consideration, for example from 0.001% to 20% of the total weight of the composition.

According to an advantageous embodiment, the combination according to the invention may be combined with one or more supplementary cosmetic active agents.

These active agents could be chosen from UV-screening agents, moisturizers, additional desquamating agents, agents for improving the barrier function, depigmenting agents, antioxidants, dermo-decontracting agents or dermo-relaxing agents, anti-glycation agents, agents for stimulating the synthesis of dermal and/or epidermal macromolecules and/or for preventing their degradation, agents for stimulating fibroblast or keratinocyte proliferation and/or keratinocyte differentiation, agents for promoting the maturation of the horny envelope, agents for increasing the activity of the sebaceous gland, agents stimulating the energy metabolism of cells and calmatives. According to a particular variant of the invention, the combination according to the invention is used together with at least one active agent chosen from UV-screening agents, antioxidants, agents for stimulating the synthesis of dermal and/or epidermal macromolecules and muscle relaxants.

These supplementary active agents may be present in the composition in a content ranging from 0.001% to 20% by weight, preferably from 0.01% to 10% by weight, and more preferentially from 0.01% to 5% by weight, relative to the total weight of the composition.

Advantageously, the composition according to the invention has a pH ranging from 3 to 7. Preferably, the pH of the composition ranges from 4 to 6.

More particularly, a subject of the invention is also a cosmetic treatment process for caring for, making up and/or cleansing the skin, comprising the application to the skin, of a composition as described above.

Said cosmetic treatment process for caring for, making up and/or cleansing the skin is non-therapeutic.

A subject of the invention is also a cosmetic process for preventing and/or treating the signs of ageing of the skin, comprising at least one step of topical application to the skin, of a composition according to the invention as described above.

The signs of ageing of the skin to be prevented and/or treated in the cosmetic process according to the invention may be chosen from wrinkles and fine lines, and/or for combating wizened, flaccid and/or thinned skin.

A subject of the invention is also the cosmetic use of a composition according to the invention as defined above, for caring for, making up and/or cleansing the skin.

The expressions “between . . . and . . . ” and “ranging from . . . to . . . ” or “at least . . . ” or “at least of . . . ” should be understood as being limits inclusive, unless otherwise specified.

The examples below of compositions according to the invention are given as illustrations with no limiting nature. The compounds are indicated as their chemical name or their INCI name.

The amounts of the ingredients are expressed as weight percentages.

### Example 1

The composition which follows was prepared according to the following procedure:

Heating the raw materials of the fatty phase at 70° C., dispersing them until complete melting is achieved. Slowly transferring the aqueous phase and dispersing it, until an emulsion in accordance with the invention is obtained with cooling to 30° C. Dispersing the active agents and the fragrance until a homogeneous product is obtained.

This composition is described in detail in Table 1 below.

TABLE 1

| | | AMOUNT |
|---|---|---|
| NAME | COMMERCIAL ORIGIN | WEIGHT % |
| DISODIUM EDTA | EDETA BD, BASF | 0.10 |
| CAPRYLOYL SALICYLIC ACID | MEXORYL SAB, CHIMEX | 0.30 |
| TRIETHANOLAMINE | TRETHANOLAMINE CARE, BASF | 0.35 |
| ACETYL | MEXORYL SAR, CHIMEX | 0.10 |
| TRIFLUOROMETHYLPHENYL | | |
| VALYLGLYCINE | | |
| SALICYLOYL | PHYTOSPHINGOSINE SLC, EVONIK | 0.0020 |
| PHYTOSPHINGOSINE | GOLDSCHMIDT | |
| RED GINSENG | RED GINSENG ACTIVE-R(PD), NATURAL | 2 |
| | SOLUTION (comprising 5% of active material) | |
| Cl 15510, monosodium salt of | UNICERT ORANGE K7011-J, SENSIENT | 0.01 |
| Orange 2 | | |
| PHENOXYETHANOL | SEPICIDE LD, SEPPIC | 0.50 |
| CAPRYLIC/CAPRIC | TRIGLYCERIDES C8C10 70/30, STEARINERIES | 3.10 |
| TRIGLYCERIDE | DUBOIS | |
| ISOPROPYL ISOSTEARATE | WICKENOL 131, ALZO | 1.20 |
| CERA ALBA | WHITE BEESWAX, KOSTER KEUNEN | 1 |
| BUTYROSPERMUM PARKII | LIPEX 102, UNIPEX | 2 |
| BUTTER | | |
| CETYL ALCOHOL | LANETTE 16, BASF | 0.50 |
| MYRISTYL MYRISTATE | TEGOSOFT MM, EVONIK GOLDSCHMIDT | 2 |
| STEARYL ALCOHOL | LANETTE 18, BASF | 0.50 |
| MICA (and) Cl 77491, MICA- | COLORONA BRONZE FINE, MERCK | 0.15 |
| BROWN IRON OXIDE | | |
| FRAGRANCE | PURE 34, GIVAUDAN | 0.25 |
| XANTHAN GUM | RHODICARE XC, RHODIA | 0.15 |
| ACRYLAMIDE/SODIUM | SIMULGEL 600, SEPPIC | 2.20 |
| ACRYLOYLDIMETHYLTAURATE | | |
| COPOLYMER (and) | | |
| ISOHEXADECANE (and) | | |
| POLYSORBATE 80 | | |
| CYCLOHEXASILOXANE | XIAMETER PMX-0246 CYCLOHEXASILOXANE, | 6 |
| | DOW CORNING | |
| GLYCEROL | ECOCEROL, ECOGREEN OLEOCHEMICALS | 7 |
| CAPRYLYL GLYCOL | HYDROLYTE CG, SYMRISE | 0.30 |
| STEARIC ACID | PALM ERA B1802CG, KLK OLEO | 3 |
| GLYCERYL STEARATE (and) | SIMULSOL 165, SEPPIC | 2 |
| PEG-100 STEARATE | | |
| PEG-20 STEARATE | MYRJ S20-PA-(MV), CRODA | 0.80 |
| DISODIUM STEAROYL | ACYLGLUTAMATE HS 21, AJINOMOTO | 0.30 |
| GLUTAMATE | | |
| TOCOPHEROL | DL ALPHA TOCOPHEROL, DSM | 0.50 |
| WATER | qs 100 | qs 100 |

This cosmetic care composition is a fresh and soft emulsion of homogeneous application, which delivers a light apricot-coloured tint to the face. This care composition is moisturizing, smoothing, firming and illuminates the skin instantly and day after day. It gives a radiant, revitalized complexion. It is suitable for all skin tones.

### Example 2

This example evaluates the desquamating activity of a combination of active agents according to the invention.

The study aims to detect the desquamating potential of the combination of active agents according to the invention in simplex solution by observation of the cohesion of the stratum corneum.

The keratolytic effect on excised skin maintained under survival conditions at 5% by weight in ethanol was evaluated. The study was carried out on viable human skin resulting from abdominal or breast reduction plastic surgery (6 donors). These skin fragments were placed in inserts, comprising a porous membrane (8 m), themselves positioned on culture wells containing a culture medium as described in Boisnic et al., Journal of Cosmetics and laser therapy, 2010:12:25-31.

The protocol consists in applying the test solutions to skin samples maintained under survival conditions. The products tested are applied in a proportion of 15 μl per 1 cm² sample and are not rinsed off. Application is carried out twice, at D0 and then, 24 hours later, at D1. The morphology of the stratum corneum is analysed 48 hours after the first application, at D2, on a biopsy.

The solutions applied are the following:
compound A (plant active agent according to the invention) alone at 0.1% and 0.5% by weight of active material in water, and
compound B alone at 0.3% by weight in water
combination of A at 0.1% and B at 0.3% by weight in water
combination of A at 0.5% and B at 0.3% by weight in water.

By way of comparison, no solution is applied to a sample, referred to as a control sample, and a solution of glycolic acid at 30% by weight in water is applied to another sample. 6 samples were used for each of the tests (controls, compound A and compound B and combinations of A and B).

The histological analysis of the horny layer is carried out on a skin section after staining with hemalaun-eosin (magnification 400). The decrease in the cohesion of the stratum corneum is expressed in the form of a score:
score 0: absence of modification
score 1: slight decrease
score 2: moderate decrease
score 3: large decrease
score 4: very large decrease with exfoliation

The results obtained appear in the Table 2 below. A paired Student test (p<0.05) was carried out in order to evaluate the significance of the difference with respect to the control.

TABLE 2

| | | | **p |
|---|---|---|---|
| | | *p | Statistical |
| | | Statistical | significant |
| | | significant | difference with |
| | Score 2: | difference with | respect to the |
| | (mean ± sd, | respect to the | skin with |
| Product | n = 6) | control skin | product B alone |
| Skin at D 0 | 0.63 ± 0.41 | — | — |
| None (control skin) | 1.30 ± 0.47 | — | — |
| Compound A at | 2.06 ± 0.43 | 0.05 | — |
| 0.1% in H₂O | | | |
| Compound A at | 2.61 ± 0.48 | 0.015 | — |
| 0.5% in H₂O | | | |
| Compound B at | 2.14 ± 0.52 | 0.030 | — |
| 0.3% in H₂O | | | |
| Compound A at | 2.47 ± 0.66 | — | 0.039 |
| 0.1% + | | | |
| Compound B at | | | |
| 0.3% in H₂O | | | |
| Compound A at | 2.91 ± 0.37 | 0.0092 | 0.035 |
| 0.5% + | | | |
| Compound B at | | | |
| 0.3% in H₂O | | | |
| Glycolic acid at | 3.10 ± 0.12 | 0.0015 | — |
| 30% in H₂O | | | |

The combinations according to the invention really do bring about a decrease in the cohesion of the stratum corneum. This decrease is statistically significant with respect to the control skin and illustrates a desquamating effect at the concentrations tested.

Moreover, the efficacy of these combinations is also significantly greater with respect to a skin treated only with compound B.

The product A at 0.5% and also the combination A at 0.5% and B at 0.300 display a desquamating effect similar to the effect obtained with 30% glycolic acid, the reference ingredient in desquamation.

### Example 3

The protocol described in Example 2 was reproduced by considering a plant active agent not in accordance with the invention, namely white ginseng which is devoid of ginsenoside Rg3. This active agent considered is the product Ginseng ES, distributed by the company Greentech.

The results are given in Table 3 below.

TABLE 3

| | Score |
|---|---|
| White ginseng | (mean ± sd, n = 6) |
| None (control) | 1.48 ± 0.14 |
| Compound A′ at 0.1% in H₂O | 2.04 ± 0.37 |
| Compound A′ at 0.5% in H₂O | 2.14 ± 0.26 |
| Compound B at 0.3% in H₂O | 1.75 ± 0.19 |
| Compound A′ at 0.1% + | 2.18 ± 0.55 |
| Compound B at 0.3% in H₂O | |
| Compound A′ at 0.5% + | 2.15 ± 0.51 |
| Compound B at 0.3% in H₂O | |
| Glycolic acid at 30% in H₂O | 2.14 ± 0.41 |

The study, carried out under conditions comparable to those considered in Example 2, shows that, by comparison with the combination of red ginseng with the compound B, the combination of white ginseng with the compound B does not reveal a greater efficacy with respect to skin treated only with compound B.

1: Composition, comprising:
from 0.001% to 10% by weight of 5-n-octanoylsalicylic acid or a salt thereof relative to the total weight of said composition
from 0.001% to 10% by weight, relative to the total weight thereof, of a plant active agent containing at least 0.05% by weight of ginsenoside Rg3 relative to the total weight of said active agent, and
water.

2: The composition as claimed in claim 1, in which said plant active agent contains at least 0.1%, by weight of ginsenoside Rg3 relative to the total weight thereof.

3: The composition as claimed in claim 1, wherein said plant active agent is red ginseng.

4: The composition as claimed in claim 1, wherein the content of 5-n-octanoylsalicylic acid or a salt thereof is from 0.05% to 2.0%, relative to the total weight of the composition.

5: The composition as claimed in claim 1, wherein the content of the plant active agent is at least 0.05% by weight.

6: The composition as claimed in claim 1, wherein a weight ratio of said plant active agent to 5-n-octanoylsalicylic acid or a salt thereof is at least equal to 0.3.

7: The composition as claimed in claim 1, wherein a content of the water is from 50% to 99% by weight relative to the total weight of the composition.

8: The composition as claimed in claim 1, wherein the composition is in the form of a water-in-oil or oil-in-water emulsion.

9: The composition as claimed in claim 1, further comprising from 1% to 80% by weight of an oily phase relative to the total weight of the composition.

10: Cosmetic treatment process for caring for and/or making up and/or cleansing the skin comprising the application to the skin, of a composition according to claim 1.

11: Cosmetic process for stimulating epidermal renewal, comprising the topical application of a composition according to claim 1.

12: Cosmetic process for promoting, restoring or increasing the homeostasis of the epidermis, comprising the topical application of a composition according to claim 1.

13: Cosmetic process for treating and/or preventing an unsightly deterioration of the complexion or of the surface of the skin, comprising the topical application of a composition according to claim 1.

14: Cosmetic process for promoting and/or increasing the hydration of the skin, comprising the topical application of a composition according to claim 1.

15: A skin care composition, comprising the composition according to claim 1.
